(19) United States
(12) Patent Application Publication
(10) Pub. No.: US 2003/0036567 A1
(43) Pub. Date: Feb. 20, 2003

(54) PROPYLENE GLYCOL AS AN AGENT TO STIMUATE THROMBOPOIESIS

(76) Inventors: Mr. Adam Asch, Mount Kisco, New York (US); Mr. Nicholas Schiff, New York, New York (US)

Correspondence Address: Darby & Darby, P.C., 805 Third Avenue, New York, New York, 10022, US

(21) Appl. No.: 10/210,854

(22) Filed: Jul. 31, 2002

Related U.S. Application Data
(60) Provisional application No. 60/309,701, filed on Aug. 2, 2001.

(51) Int. Cl. A61K 38/20; A61K 38/19; A61K 31/045

(52) U.S. Cl. 514/738; 424/85.2; 424/85.1

(57) ABSTRACT

### Abstract of Disclosure

Propylene glycol can be administered therapeutically to effectively reverse thrombocytopenia and increase platelet cell counts, particularly in patients suffering from medical conditions such as HIV/AIDS or undergoing chemotherapy.

### Cross Reference to Related Applications

This application claims priority under 35 U.S.C. §119(e) of provisional application Serial No. 60/309,701, filed August 2, 2001, which is incorporated herein by reference in its entirety.

### Federal Research Statement

This application was supported by in part by NIH grant R01 DK48698. Pursuant to the terms of the grant the federal government may have certain rights to this invention.

### Background of Invention

This invention relates to therapies employing propylene glycol, and especially those which include the treatment of thrombocytopenia in immunodeficient and chemotherapy patients.

Platelet Disorders. Hematopoietic differentiation into the myeloid pathway follows three branches, erythroid, megakaryocytic and phagocytic. Platelets, which play a critical role in blood coagulation, wound healing and the storage and release of cytokines, are produced by megakaryocytosis. A portion of the platelets are sequestered in the spleen; the remaining platelets circulate in the bloodstream, and under normal conditions are replenished as needed by the megakaryocyte, a process regulated by thrombopoietin (TPO). TPO binds to platelets; when platelet numbers are reduced, the increased level of free TPO signals megakaryocyte and platelet production. Thrombocytopenia, depletion of the circulating level of platelets to less than 100,00/mL, is caused by one of three mechanisms: decreased bone marrow production, increased splenic sequestration, and accelerated platelet destruction. Radiation therapy, cytotoxic chemotherapeutic agents, surgery, accidental blood loss, and other specific disease conditions depress megakaryocyte production and may cause thrombocytopenia.

Methods for stimulating platelet levels in patients undergoing treatments such as chemotherapy, without altering the dosing or interrupting the timing of such treatments due to low platelet counts, have been developed. However many of these therapies are associated with severe side effects, or are not yet publicly available. NEUMEGA (oprelvekin; Wyeth), a human recombinant IL-11, is indicated for the prevention of severe thrombocytopenia. Administration of NEUMEGA has been found to reduce the need for platelet transfusions following myelosuppressive chemotherapy in patients with nonmyeloid malignancies who are at high risk of severe thrombocytopenia. However, this agent has adverse side effects including peripheral edema, dyspnea, tachycardia, and conjunctival redness and is not recommended for use in patients with or at risk of congestive heart failure. GM-CSF, approved by the FDA for use in patients undergoing high dose chemotherapy and for bone marrow transplantation, causes eosinophilia. TPO for treatment of thrombocytopenia associated with cancer chemotherapy, and megakaryocyte growth differentiation factor polypeptide attached to polyethylene glycol, are both completing clinical trials.

Propylene glycol. Propylene glycol is a widely available organic solvent which the Food and Drug Administration has classified as "generally recognized as safe," i.e., it is acceptable for use in flavorings, drugs, and cosmetics, and as a direct food additive. Marketing of propylene glycol as an environmentally friendly antifreeze makes large quantities of this solvent readily available, leading to accidental or intentional propylene glycol poisoning or intoxication. Because it is typically thought to be pharmacologically inert, propylene glycol overdose is not usually considered in the differential diagnosis of acute large anion gap acidosis or included in standard toxicological studies. However, because propylene glycol is widely used as a solvent for oral and intravenous drugs, poisoning or intoxication can also occur in subjects undergoing medical treatment or therapy. Consequently, propylene glycol toxicity occurs in intravenous infusion treatments (Demey, HE, Daelemans RA, De Bro, ME, Bossaert, L. Propyleneglycol intoxication due to intravenous nitroglycerin. Lancet 1984; 1:1360 Demey HE, Daelemans RA, Verpooten GA, et al. Propylene glycol induced side effects during intravenous nitroglycerin therapy. Intensive Care Med 1988; 14:221-6).

Typically, propylene glycol poisoning causes a number of neurological and systemic abnormalities in humans, including central nervous system depression, tachyarrthymias, hypotension, metabolic acidosis, renal failure, and hematologic dysfunction (Martin, G., Finberg, L. Propylene glycol: a potentially toxic vehicle in liquid dosage form. Journal of Pediatrics 1970; 77(5): 877-878; Arulanantham, K., Genel, M. Central nervous system toxicity associated with ingestion of propylene glycol. Journal of Pediatrics 1978; 93:515-516.). Denning DW and Webster AD (J Pharm Pharmacol, 1987, 39(3): 236-8) disclose that propylene glycol at a concentration of 0.5-1.0% , at or below the standard concentration in pharmaceuticals, demonstrated immunosuppressive effects. Morshed KM et al. (Toxicol Sci, 1998, 46(2): 410-7) disclosed toxic effects of propylene glycol at 50mM observed in normally proliferating cultured human proximal tubule (HPT) cells. Morshed cautioned that mild subacute toxicity occurred at concentrations that could be achieved in human plasma when HPT were used as a drug vehicle.

Alterations in hematologic parameters attributable to propylene glycol administration are also described. Saini M et al. (Vet Hum Toxicol, 1996, 38(2):81-5) describes that reticulocyte counts, plasma hemoglobin and osmolality from a single oral dose of propylene glycol decreased and then increased, and the integrity of erythrocytes was transiently disrupted. The article suggests that these changes, and an observed significant increase in spleen weight, were due to removal of damaged cells from the bloodstream. Bauer MC et al. (Vet Hum Toxicol, 1992, 34(2):127-31) describes accelerated erythropoiesis and reduction in red blood cell life span in kittens fed a diet containing 6 or 12% propylene glycol. Cate and Hedrick reported a case (NEJM 11/1980) of accidental intoxication which presented with acute large anion gap acidosis (A.G.=28) and stupor. A recent chart review of this case revealed the presence of leukocytosis, with an elevated WBC of 25,000 (per mm³). A study of bone marrow of lithographers exposed to a dipropylene glycol monomethyl ether after one worker developed an aplastic anemia revealed a generally hypocellular marrow with decreased myeloid to erythroid ratios and an abundance of stored iron, with moderate eosinophilia and prominent mast cells (Cullen, MR, Rado, T., Waldron, JA, Sparer, J. and Welch, L. Bone marrow injury in lithographers exposed to glycol ethers and organic solvents used in multicolor offset and ultraviolet curing printing processes. Archives of Environmental Health 1983; 38(6): 347-354).

It is desirable to develop alternative treatments to treat platelet disorders and to reverse the effects of thrombocytopenia using materials that are less costly than the existing regimens, that advantageously avoid the accompanying side effects of known treatments, and are therapeutically equivalent to therapeutic agents such as those used for chemotherapy. The present invention provides such an alternative treatment using propylene glycol as a therapeutic agent for improving hematopoietic stem cell proliferation, particularly, in conditions where such proliferation would facilitate recovery after bone marrow transplant or chemotherapy.

### Summary of Invention

The present invention provides a method of increasing platelet counts in a patient in need thereof. The method comprises administering to the patient a therapeutic composition comprising propylene glycol and a pharmaceutically acceptable carrier. The composition may be administered in a therapeutically effective amount and for a time sufficient to increase platelet cell counts to normal or nearnumbers in a thrombocytopenic patient. The therapeutic amount is sufficient to increase platelet cell counts in the patient above baseline within several days of administration. Patients in need of such treatment include those who are afflicted with immune disorders or platelet disorders. The present method is particularly effective in a patient that has undergone chemotherapy or radiation therapy. It is also contemplated that the method of the invention maybe used in patients who are immunodeficient and patients who have HIV/AIDS. In a preferred embodiment, the present invention provides a method for treating a patient suffering from or at risk of thrombocytopenia. The method comprises administering to the patient an amount of propylene glycol sufficient to increase the level of formation of platelets or their progenitors.

According to the method of the invention, the composition may be administered with a cytokine selected from the group consisting of erythropoietin (EPO), granulocytefactor (Gcolonyfactor (CSFgranulocytecolonyfactor (GMinterleukin(ILinterleukin(ILinterleukin(ILinterleukin(ILinterleukininterleukin(ILmegakaryocytecell factor (megand megakaryocytefactor (M The cytokine may be administered either in the same composition or co-administered, i.e., in separate preparations as part of the same therapeutic regimen.

The propylene glycol is administered orally or intravenously to achieve a serum concentration of 1pM to 1nM.

In a further embodiment, the present invention provides a method for enhancing proliferation or differentiation of hematopoietic stem cells or progenitor cells. The method comprises exposing the cells to propylene glycol in an amount effective for enhancing proliferation or differentiation of the cells. In one embodiment, the cell is a CD34+CD38- hematopoietic stem cell. The hematopoietic cell maybe in cell culture or may be present in an animal. The animal may be a mammal, and preferably is a human.

### Brief Description of Drawings

Figure 1 shows the effect of propylene glycol on the formation of colonies from CD34+CD38-hematopoietic stem cells.

Figure 2 shows the effect of propylene glycol in the long term culture initiating cell assay (LTCIC).

### Detailed Description

The present invention is based in part on the observations that propylene glycol intoxication resulted in dramatic hematologic changes, previously unrecognized as connected to exposure to large amounts of propylene glycol. Applicants have discovered that administration of controlled dosages of propylene glycol may be beneficial in modulating various hematopoietic parameters and immune deficiencies.

The present invention involves the discovery that propylene glycol may be beneficial in stimulating hematopoiesis. This discovery is based, in part, on clinical studies and laboratory analysis of samples from patients who had propylene poisoning or intoxication. Laboratory analysis of the blood of a patient who had propylene glycol poisoning (blood levels of propylene glycol at least as high as 2mM after ingestion) showed a dramatic increase in leukocytosis and thrombocytosis in response to acute exposure to propylene glycol. A bone marrow biopsy performed about 1 month after the ingestion of propylene glycol revealed increases in myeloid precursors and megakaryocytes.

The discovery is also based on in vitro data which demonstrated that propylene glycol increased the formation of colonies from CD34+CD38- hematopoietic stem cells, with maximum effect seen at a propylene glycol concentration of from 50 to 100pM.

The term propylene glycol as used herein includes propylene glycol and its analogs, esters and ethers, ethylene glycol and glycerol, polyethylene glycol esters and ethers, and polypropylene esters and ethers. All these compounds are generally recognized as safe and widely used in the pharmaceutical and food industries.

According to the invention, propylene glycol may be used in a pharmaceutical composition or formulation to stimulate platelet recovery in thrombocytopenic patients, including HIV/AIDS and chemotherapy patients. Patients suffering from thrombocytopenias associated with marrow hypoplasia, e.g., aplastic anemia following bone marrow transplantation or adverse side effects of various drugs can also benefit by way of the methods and compositions of the present invention. Other conditions characterized by decreased platelet counts would also benefit by way of the present invention.

By suppressing or preventing thrombocytopenia, that has been observed very early with the onset of HIV infection in a number of HIV patients, one may well advantageously be able to lengthen the time between HIV infection and the onset of AIDS symptoms. By providing a treatment for thrombocytopenia in HIVpatients, the compositions and methods of the present invention may unexpectedly and advantageously provide for a significant delay in the onset of AIDS.

Within the scope of the invention is the treatment of disorders resulting from defects in platelets or damage to platelets, e.g., resulting from transient poisoning of platelets by other chemical or pharmaceutical agents or therapeutic manipulations.

Therapeutic treatment of such thrombocytopenic disorders or deficiencies with the compositions of the present invention can avoid undesirable side effects which result from treatment with presently available serumfactors or transfusions of human platelets, which may pose serious health risks.

The dosage regimen for treating the aboveconditions by way of the present invention will be determined by the attending physician giving consideration to various factors, such as, the condition, the body weight, the sex and diet of the patient, the severity of any infection, time of administration and other clinical factors. The propylene glycol may be administered over a period of time sufficient for therapeutically increasing and maintaining platelet cell count numbers. Progress of the treated patient, as measured by an increase in platelet cell counts, can be readily monitored by standard techniques.

The therapeutic composition can be administered orally or parenterally by means well known to one of skill in the art for such administrations. Particularly, the composition may be administered intravenously. If desirable, the composition may be also administered subcutaneously. When systemically administered, the therapeutic composition is in the form of a pyrogenparenterally acceptable aqueous solution. The preparation of such pharmaceutically acceptable solutions, having due regard to pH, isotonicity, stability, and the like, is within the skill of the art.

The therapeutic methods and compositions of the present invention may also be employed, alone or in combination, with other cytokines, hematopoietins, interleukins, growth factors, or antibodies in the treatment of disease states characterized by other symptoms of other cellular deficiencies as well as platelet deficiencies. Other megakaryocytic stimulatory factors, e.g., megor other molecules with TPOactivity may also be employed with propylene glycol. Additional exemplary cytokines or hematopoietins for such coinclude CSFILILILMand ILThe dosage recited above for propylene glycol would be adjusted accordingly to compensate for any additional components in the therapeutic composition.

Disease conditions that may be treated in accordance with the present invention include aplastic anemia, cytopenia secondary to chemotherapy for malignant disease, panocytopenia due to myelodyplastic syndrome, and metastatic tumors which result in thrombocytopenia. Conditions found in certain treatments for AIDS which result in thrombocytopenia (e.g., AZT) and certain wound healing disorders may also benefit from an increase in platelet numbers and thus may be treated in accordance with the present invention.

With regard to anticipated platelet deficiencies, e.g., due to future surgery, propylene glycol could be administered several days to several hours prior to the need for platelets. With regard to acute situations, e.g., accidental and massive blood loss, propylene glycol could be administered along with blood or purified platelets.

Such compositions comprise a therapeutically effective amount of propylene glycol in admixture with a pharmaceutically acceptable carrier. The carrier material may be water for injection, preferably supplemented with other materials common in solutions for administration to mammals. Typically, propylene glycol will be administered in conjunction with one or more physiologically acceptable carriers, excipients, or diluents. Neutral buffered saline or saline mixed with serum albumin are exemplary appropriate carriers. Other standard carriers, diluents, and excipients may be included as desired. Other exemplary compositions are Tris buffer, pH 8.0 and acetate buffer, pH 5.0, which, in each case, may further include sorbitol. The present compositions can be systemically administered parenterally. Alternatively, the compositions may be administered intravenously or subcutaneously. When systemically administered, the therapeutic compositions for use in this invention may be in the form of a pyrogenparenterally acceptable aqueous solution. The preparation of such pharmaceutically acceptable protein solutions, with due regard to pH, isotonicity, stability and the like, is within the skill of the art.

The following examples illustrate the invention without limitation.

The following are case reports of two patients with proven propylene glycol intoxication. In both cases several unrecognized complications of acute and chronic exposure to this compound were observed. Both patients suffered profound central nervous system compromise and one patient developed cardiovascular collapse with marked hematological changes including leukocytosis, thrombocytosis, microcytic anemia and bone marrow abnormalities.

Example 1. Patient 1 was a 32-year-old woman with a history of congenital aqueductal stenosis diagnosed at age fourteen and treated with a Torkildson shunt. Nineteen months prior to admission, she sustained a minor head injury in a motor vehicle accident resulting in a shunt malfunction with recurrent headaches, necessitating treatment with a ventriculoperitoneal shunt. Consequent subdural hygromas became infected, leaving the patient with marked cognitive and motor impairments. She was transferred to a rehabilitation facility six months prior to admission to our hospital. While in rehabilitation, the patient had an acute onset of tachycardia (150 bpm), hyperpyrexia (103.9), tachypnea, and a grand mal seizure. Laboratory examination at this time revealed a white blood cell count of 28,000 (per mm³), a thrombocyte count of 777,000 (per mm³) a Hgb of 11.1 (g/dl), a Hct of 36.5 (%), a serum sodium of 135 (mmole/liter), a potassium of 4.6 (mmole/liter), a chloride of 97 (mmole/liter), and bicarbonate of 27 (mmole/liter) with elevated anion gap (A.G.=14). Urinalysis revealed protein of 50 (mg/dl); the presence of hyaline casts and blood were noted. Urine culture showed 80,000 CFU of E. faecalis and she was treated for a urinary tract infection. All biochemical and hematologic abnormalities resolved within 36 hours. The patient remained well until four weeks prior to admission when she again developed fever (106), tachycardia (169 bpm), and tachypnea. Similar biochemical and hematological abnormalities were noted including a white blood cell count of 25,000 (per mm³) and a thrombocyte count of 469,000 (per mm³). Cultures of blood, urine, and CSF were negative. Within 36 hours the patient returned to baseline. Similar episodes were noted to occur at roughly seven day intervals. CT scans of chest, abdomen and pelvis, and a gallium scan were unremarkable. After the fourth recorded episode, the patient was transferred to our neurological unit for evaluation of an occult ventriculoperitoneal shunt infection or possible hypothalamic dysregulation.

On transfer, the patient was noted to have BP 120/80, HR 88 bpm and T 37.5. The general physical examination was notable only for generalized pallor and contractures of the lower extremities. She was awake but only able to follow single midline one-step commands. She had a depressed gag reflex bilaterally, quadriparesis, diffuse hyperreflexia, and bilateral extensor plantar responses. Initial laboratory evaluation revealed a WBC of 5800 (per mm³), platelet count of 379,000 (per mm³), a Hgb of 7.2 (g/dl), a Hct of 21.6 (%), sodium of 133 (mmol/liter, potassium of 4.4 (mmol/liter), chloride 28 (mmole/liter) and bicarbonate of 28 (mmole/liter) with anion gap of 1. CSF examination revealed an acellular fluid with protein of 35 (mg/dl) and glucose of 49 (mg/dl). Cultures of CSF, blood, and urine were negative. A head CT scan with and without iodinated contrast revealed marked lateral ventricular dilatation and no abnormally enhancing regions. No clinical change was noted for three days. On the night prior to returning to the rehabilitation facility, the patient exhibited tachycardia (170 bpm), hypotension (90/60 mmHg), tachypnea, fever of 39.5, and rhythmic tonic-clonic movements of her upper and lower extremities. Laboratory analysis revealed a white blood cell count of 32,000 (per mm³), thrombocyte count of 724,000 (per mm³), a sodium of 141 (mmole/liter), a potassium of 5.8 (mmole/liter), a chloride of 101 (mmole/liter), a bicarbonate of 16 (mmole/liter) a large anion gap (A.G.=24), a LDH of 528 (U/liter), a SGOT of 151 (U/liter), a creatinine kinase of 8400 (U/liter) and an arterial blood gas of pH 7.36, pCO₂25 (mmHg) and pO₂95 (mmHg). The patient was treated with kayexalate. The anion gap acidosis resolved within twelve hours. At that time, the patient was noted to develop oliguric renal insufficiency with a BUN of 41 (mg/dl) and creatinine of 2.2 (mg/dl). Urinalysis revealed large amounts of blood and the presence of hyaline and granular casts. An unusual "green stool" which caked the patient's rectum was collected. Samples of the patient's blood, urine, and stool (collected 17 hours after the recorded onset of symptoms) were sent for toxicologic analysis.

National Medical Services (Willow Grove, PA) performed assays for propylene glycol by gas chromatography (GC) with ionization detection, using a direct injection method. The laboratory confirmed the identity of propylene glycol by gas chromatography with mass spectrometry (GCMS).

A bone marrow biopsy revealed a hypercellular marrow with reactive features including an increased myeloid:erythroid ratio and an increase in earlier myeloid precursors. In addition, prominent throughout the aspirate were eosinophils, mast cells as well as eo-basophils. Megakaryocytes were abundant.

Because of suspected deliberate poisoning, the patient remained under guard for the rest of her hospitalization. She was noted over a period of 6-8 weeks to have marked improvement in her mental status as evidenced by production of spontaneous speech, ability to feed herself, reorientation to person, place and elements of her autobiographical history. Over the same time course, the patient's anemia also began to resolve.

The patient demonstrated a dramatic leukocytosis and thrombocytosis in response to the acute exposure to propylene glycol and long term displayed a reticulopenia that resolved over several weeks. Bone marrow studies performed four weeks after acute exposure revealed a relatively hypercellular marrow with increased myeloid precursors and megakaryocytes. There was abundant stored iron, as well as an increase in eosinophils and basophils. In addition, the patient displayed frequent eo-basophils throughout the marrow.

Example 2. A two and one-half year old female ingested an unknown amount of the antifreeze Freeze Proof containing 30% propylene glycol earlier. Following ingestion, the child acted normally and ate well until approximately 6 hours later when she developed slurred speech, right facial drooping, an abnormal, wobbling gait, and was noted walking into walls and unable sit up by herself. She was the brought to the emergency room approximately 12 hours after ingestion with the above symptoms. At that time a toxicological screen was obtained which was qualitatively positive for propylene glycol. She was then transferred to University Medical Center where her blood pressure was 106/70, pulse was 113, respiration was 18, temperature was 36.3 C. At that time she had slurred speech, good facial symmetry, intact cranial nerve examination, but an unsteady gait, and was drowsy and lethargic but arousable. Her pupils were equal and reactive to light. The laboratory examination showed a sodium of 137, potassium of 3.6, chloride of 108, bicarbonate of 22, anion gap of 7, glucose of 130, BUN of 11, creatinine of 0.3, white blood cell count of 10,100, red blood cell count of 3,460,00, hemoglobin 10.1, hematocrit 28.6, mcv 82.6, and a platelet count of 278,000. The results also showed 69.6 segmented neutrophils, 24.4 lymphs, 5 monocytes, an occasional eosinophil and basophils. Blood gases showed a pH of 7.35, a pCO₂ of 30, pO₂ of 101, bicarbonate of 16 and oxygen saturation of 97. The child was given a charcoal and sorbitol lavage through an oral naso-gastric tube as well as IV fluid therapy with normal saline. Within 4 hours of admission, the child was able to walk and talk well without evidence of slurred speech. The following morning the child was discharged from the hospital with no evidence of the symptoms originally observed.

Discussion. The identification of a large anion gap metabolic acidosis in patient with an unidentified and unusual illness should alert the physician to the possibility of deliberate intoxication. We herein have described two patients with acute propylene glycol intoxication, with chronic exposure in the case of Patient 1. Patient 1 experienced several unreported manifestations of propylene glycol intoxication including remarkable acute elevation of the white blood cell and thrombocyte counts, chronic bone marrow toxicity, and chronic CNS depression. Patient 2 had the acute onset of multifocal neurological signs that resolved quickly with treatment and led to no biochemical or hematological abnormalities. These cases illustrate the serious nature of both the neurological and systemic effects of propylene glycol intoxication including tachyarrthymias, hypotension, hyperkaliema, metabolic acidosis, oliguric renal failure and hematologic dysfunction. The widespread availability of this compound and advertising claims of low toxicity suggests that more such cases will arise.

Because it distinguishes among 1,2 -ethanediol (ethylene glycol), 1,2-propanediol (propylene glycol), 1,3-propanediol, and 1,2-butanediol, gas chromotography (without mass spectrometry) is specific enough for routine clinical use. Importantly, 1,2-propanediol (propylene glycol) is frequently used as an internal standard in gas chromatographic analysis of ethylene glycol (included in all toxicologic screenings of large anion gap acidoses). Thus, because of its expected presence in the assay, elevated concentrations are likely to escape notice or be attributed to an overly generous sample size. Initial evaluation of Patient 1's samples led to exactly this confusion. If 1,3-propanediol or 1,2-butanediol is used as the internal standard, gas chromatography identifies and quantifies both ethylene glycol and propylene glycol simultaneously.

Example 3. CD34+CD38- cells isolated from human bone marrow (Poietic Technologies) were counted using a hemocytometer and plated in semi-solid culture media (MethoCult GF+H4435, StemCell Technologies, Vancouver, British Columbia) containing methylcellulose, fetal bovine serum, BSA, recombinant human (rh) SCF, rh GM-CSF, rh G-CSF, rh IL-3, rh IL-6, rh erythropoietin, and L-glutamine to which were added varying concentrations of propylene glycol (FW=76.1 Density=1.04g/ml Fisher Scientific). The cells were plated at a density of 500 cells per 1 ml per 35 mm petri dish. After incubation at 37ºC, 5% CO₂ for 14 days, the dishes were scored for colony formation.

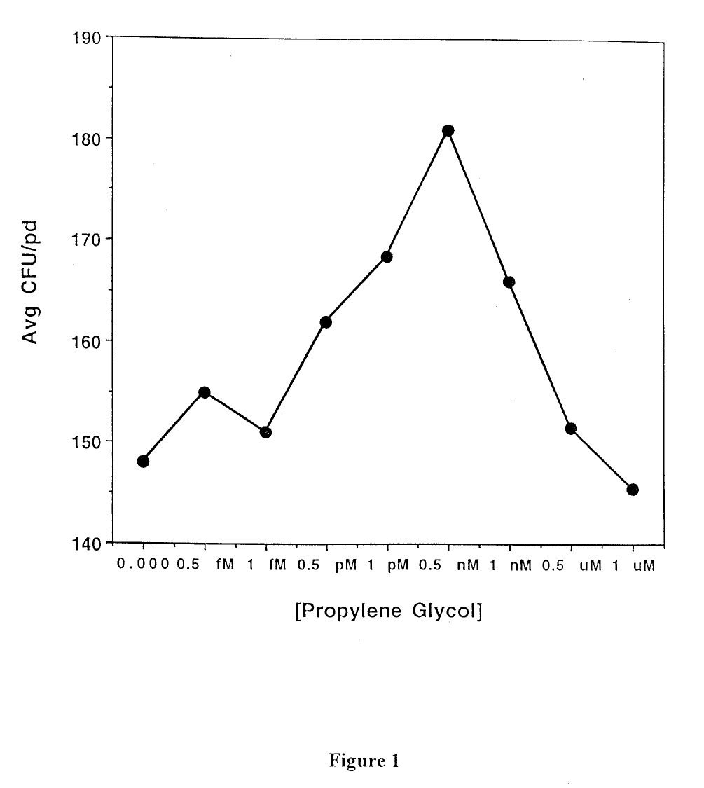

The results shown in Figure 1 demonstrate that propylene glycol increased CFU formed from CD34+ cells with maximum effects seen at a concentration of from 50 to 100 pM propylene glycol.

Example 4. Murine bone marrow was obtained from the femurs of adult C57bl mice, suspended in Myelocult (Stem Cell Technologies, Vancouver Canada), and cells were isolated by centrifugation at 500g for 10 minutes at 4 ºC. Viability was determined by Trypan blue exclusion and aliquots were taken for plating efficiency in methylcellulose culture (MethoCult, Stem Cell Technologies). The remaining cell suspension was plated at a density of 6.25 x10⁴/well into 24 well plates containing irradiated FBMD-1 feeder layers. To appropriate wells, propylene glycol was added to final concentration of 500 ng/ml. At weekly interval the cells were demidepopulated and fed with 250μl fresh medium. The harvested cells were washed with PBS and added to a 2.5 ml tube of MethoCult. Two 35mm petri dishes containing 1 ml each were plated from each tube. After 10 days incubation at 37ºC, 5% CO₂, the dishes were scored for colonies.

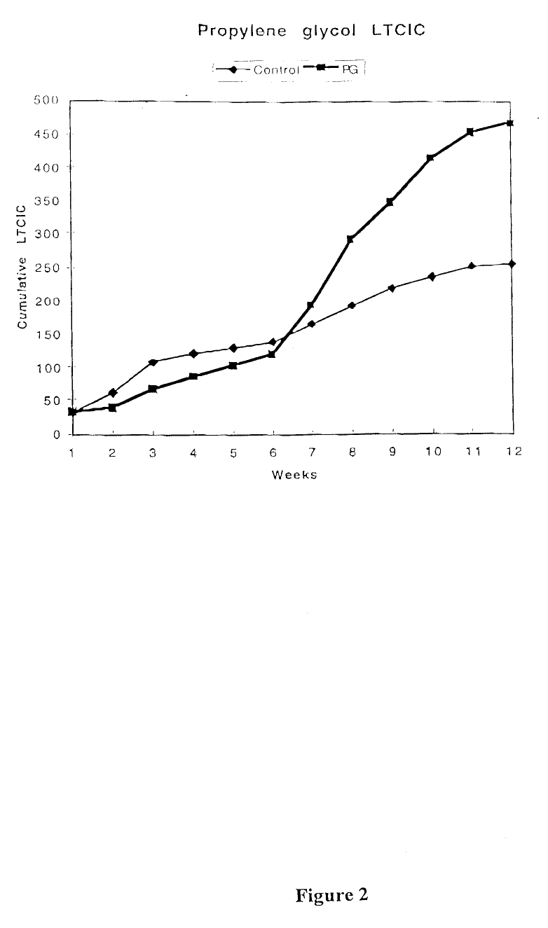

The results shown in Figure 2 demonstrate that propylene glycol increased total cell number on the cultures by 50% over those cells not exposed to propylene glycol.

We investigated the direct effects of propylene glycol on the formation of colonies from CD34+C38- hematopoietic stem cells. Propylene glycol increased CFU formed from CD34+ cells with maximum effects seen from 50 to 100 pM propylene glycol. In patient 1, the blood levels of propylene glycol were acutely greater than 2mM, but the effects on hematopoiesis were observed at a time when these levels were likely to have been much lower. The stimulatory effects of propylene glycol are of interest given its use as a solvent in injectable pharmacologic agents and might have played a role in the drastic thrombocytosis in this patient seen acutely with each toxic exposure. Based on the foregoing, it is believed that propylene glycol has a direct effect on the differentiation and proliferation of hematopoietic progenitors.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The disclosures of any patents, patent applications, and publications cited herein are incorporated by reference in their entireties.

Claims

1.A method of increasing platelet counts in a patient in need thereof which comprises administering to the patient a therapeutic composition comprising propylene glycol and a pharmaceutically acceptable carrier, said therapeutic amount being sufficient to increase platelet cell counts in said patient above baseline within several days administration.

2.The method of claim 1 wherein the patient has undergone chemotherapy or radiation therapy.

3.The method of claim1 wherein the patient has HIV/AIDS.

4.The method of claim 1 wherein the patient is immunodeficient.

5.The method of claim 1 wherein the propylene glycol is administered orally or intravenously.

6.The method of claim 1 wherein the propylene glycol is administered in an amount to achieve a serum concentration of 1pm to 1nM.

7.The method of claim 1 wherein the composition is administered with a cytokine selected from the group consisting of erythropoietin (EPO), granulocytefactor (Gcolonyfactor (CSFgranulocytecolonyfactor (GMinterleukin(ILinterleukin(ILinterleukin(ILinterleukin(ILinterleukininterleukin(ILmegakaryocytecell factor (megand megakaryocytefactor (M-CSF).

8. A method for treating a patient suffering from or at risk of thrombocytopenia, comprising administering to the patient an amount of propylene glycol sufficient to increase the level of formation of megakaryocytes or their progenitors.

9.The method of claim 8 wherein the propylene glycol is administered orally or intravenously.

10.The method of claim 8 wherein the propylene glycol is administered in an amount to achieve a serum concentration of 1pm to 1nM.

11.A method for enhancing proliferation or differentiation of hematopoietic stem cells or progenitor cells comprising exposing the cells to propylene glycol in an amount effective for enhancing proliferation or differentiation of the cells.

12.The method of claim 11 wherein the cell is a CD34+CD38- hematopoietic stem cell.

13.The method of claim 11 wherein the cell is in cell culture.

14.The method of claim 11 wherein the cell is present in an animal.

15.The method of claim 14 wherein the animal is a mammal.

16.The method of claim 15 wherein the animal is a human.
